SEQUENCE LISTING (5 sequences)

SEQ ID NO: 1 - protein, 406 aa, human coagulation Factor VII
  MISC_FEATURE (1)..(406): Xaa means 4-carboxyglutamic acid (gamma-carboxyglutamate)
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - DNA, 56 bp, Artificial
  DNA oligonucleotide primer
ggcgtcctgcaccggcgccggcgcatttcttacagtgatggggaccagtgtgcctc

SEQ ID NO: 3 - DNA, 56 bp, Artificial
  DNA oligonucleotide primer
gaggcacactggtccccatcactgtaagaaatgcgccggcgccggtgcaggacgcc

SEQ ID NO: 4 - DNA, 54 bp, Artificial
  DNA oligonucleotide primer
ggcgtcctgcaccggcgccggcgcacgcacaaggatgaccagctgatctgtgtg

SEQ ID NO: 5 - DNA, 54 bp, Artificial
  DNA oligonucleotide primer
cacacagatcagctggtcatccttgtgcgtgcgccggcgccggtgcaggacgcc

(19) United States
(12) Patent Application Publication
(10) Pub. No.: US 2007/0037746 A1
(43) Pub. Date: Feb. 15, 2007

(54) Novel compounds

(75) Inventors: Henrik Ostergaard, Roskilde (DK); Egon Persson, Ballerup (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/497,072

(22) Filed: Aug. 1, 2006

Related U.S. Application Data
(63) Continuation of application No. PCT/EP05/50474, filed on Feb. 3, 2005.

(30) Foreign Application Priority Data
Feb. 3, 2004 (DK) PA 2004 00160
Jun. 29, 2004 (DK) PA 2004 01018

(51) Int. Cl. A61K 38/37; C07K 14/75

(52) U.S. Cl. 514/12; 530/383

(57) ABSTRACT

The present invention relates to novel coagulation factor FVIIa polypeptides.

### CROSS-REFERENCE TO RELATED PATENT APPLICATIONS

This patent application is a continuation of International (PCT) Patent Application PCT/EP2005/050474 (published as WO 2005/075635), filed on Feb. 3, 2005, and claims the benefit of U.S. Provisional Patent Application 60/542,989 and 60/587,342, filed Feb. 9, 2004 and Jul. 13, 2004, respectively, and Danish Patent Applications PA 2004 00160 and PA 2004 01018, filed Feb. 3, 2004 and Jun. 29, 2004, respectively, the entirety of each of which being hereby incorporated by reference.

### FIELD OF THE INVENTION

The present invention relates to novel human coagulation Factor VII/VIIa proteins having coagulant potential/activity as well as pharmaceutical compositions comprising the polypeptides, uses and methods of treatment.

### BACKGROUND OF THE INVENTION

Blood coagulation is a process consisting of a complex interaction of various blood components (or factors) that eventually gives rise to a fibrin clot. Generally, the blood components, which participate in what has been referred to as the coagulation “cascade”, are enzymatically inactive proteins (proenzymes or zymogens) that are converted to proteolytic enzymes by the action of an activator (which itself is an activated clotting factor). Coagulation factors that have undergone such a conversion are generally referred to as “active factors”, and are designated by the addition of the letter “a” to the name of the coagulation factor (e.g. Factor VIIa).

Initiation of the haemostatic process is mediated by the formation of a complex between tissue factor, exposed as a result of injury to the vessel wall, and Factor VIIa. This complex then converts Factors 1× and X to their active forms. Factor Xa converts limited amounts of prothrombin to thrombin on the tissue factor-bearing cell. Thrombin activates platelets and Factors V and VIII into Factors Va and VIIIa, both cofactors in the further process leading to the full thrombin burst. This process includes generation of Factor Xa by Factor IXa (in complex with factor Villa) and occurs on the surface of activated platelets. Thrombin finally converts fibrinogen to fibrin resulting in formation of a fibrin clot. In recent years Factor VII and tissue factor have been found to be the main initiators of blood coagulation.

Factor VII is a trace plasma glycoprotein that circulates in blood as a single-chain zymogen. The zymogen is catalytically inactive. Single-chain Factor VII may be converted to two-chain Factor VIIa by Factor Xa, Factor XIIa, Factor IXa, Factor VIIa or thrombin in vitro. Factor Xa is believed to be the major physiological activator of Factor VII. Like several other plasma proteins involved in haemostasis, wild type Factor VII is, like a number of other coagulation proteins, dependent on Vitamin K for its activity, which is required for the gamma-carboxylation of multiple glutamic acid residues that are clustered close to the amino terminus of the protein. These gamma-carboxylated glutamic acids are required for the calcium ion-induced interaction of wild type Factor VII with phospholipids. The conversion of zymogen Factor VII into the activated two-chain molecule occurs by cleavage of an internal Arg152-Ile153 peptide bond. In the presence of tissue factor, phospholipids and calcium ions, the two-chain Factor VIIa rapidly activates Factor X or Factor IX by limited proteolysis.

Thus, wild type Factor VII has a domain structure comprising a domain rich in gamma-carboxyglutamic acid residues (the “GLA domain”), a region containing sequences homologous to human epidermal growth factor, and a catalytic domain containing a serine protease catalytic triad. The catalytic domain is glycosylated in nature.

It is often desirable to stimulate or improve the coagulation cascade in a subject. Factor VIIa has been used to control bleeding disorders that have several causes such as clotting factor deficiencies (e.g. haemophilia A and B or deficiency of coagulation Factors XI or VII) or clotting factor inhibitors. Factor VIIa has also been used to control excessive bleeding occurring in subjects with a normally functioning blood clotting cascade (no clotting factor deficiencies or inhibitors against any of the coagulation factors). Such bleeding may, for example, be caused by a defective platelet function, thrombocytopenia or von Willebrand's disease. Bleeding is also a major problem in connection with surgery and other forms of tissue damage.

FVII can be prepared recombinantly, but the primary structure of wild type Factor VII renders production of the functional protein in prokaryotic host cells impossible, since bacteria do not have the capacity to introduce the vitamin K-dependent gamma-carboxylation essential to membrane binding of the protein. Traditionally, production of wild type FVII is restricted to expression in higher, mammalian cells. However, expression in mammalian cells is much more complicated and time-consuming than expression in prokaryotes, and the yields are as a rule more limited; in general production in mammalian cells is therefore more expensive than production using prokaryotic host cells.

Wild type FVIIa, in contrast to other, homologous serine proteases, possesses an active conformation that is energetically unfavorable. The consequence is a far from optimal enzymatic activity of free wild type human Factor VIIa, which is dramatically enhanced upon binding to the cognate, membrane-bound cofactor tissue factor (TF). In the natural environment, the zymogenicity of free wild type human Factor VIIa ensures timely triggering and appropriate location of FVIIa haemostatic activity upon vascular lesion and concomitant TF exposure.

European Patent No. 200,421 (ZymoGenetics) relates to the nucleotide sequence encoding human Factor VII and the recombinant expression of Factor VII in mammalian cells. Dickinson et al. (Proc. Natl. Acad. Sci. USA (1996) 93, 14379-14384) relates to a Factor VII variant wherein Leu305 has been replaced by Ala (FVII(Ala305)).

Iwanaga et al. (Thromb. Haemost. (supplement August 1999), 466, abstract 1474) relates to Factor VIIa variants wherein residues 316-320 are deleted or residues 311-322 are replaced with the corresponding residues from trypsin. Sakai et al. (J. Biol. Chem. 1990; 265:1890-1894) and Nicolaisen et al. (FEBS Lett. 1992; 306:157-160) relates to the GLA domain of FVIIa.

Published international patent applications WO 01/83725, WO 02/22776, WO 03/027147, WO 03/037932, and WO 04/029090 all relate to variants of Factor VIIa with preserved or increased activity. WO 02/077218 relates to derivatives of Factor VIIa with prolonged serum half-life.

### SUMMARY OF THE INVENTION

It is an object of the invention to provide for less expensive, easier to produce, proteins having substantially the same or increased proteolytic activity compared to recombinant wild type human Factor VIIa.

The present invention relates in a broad aspect to a FVII polypeptide with substantially the same or increased proteolytic activity compared to recombinant wild type human Factor VIIa, wherein the FVII polypeptide is essentially free of a functional lipid membrane binding domain.

In a first aspect the present invention relates to a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues.

In a second aspect the present invention relates to a polynucleotide construct encoding a Factor VII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues.

The term “construct” is intended to indicate a polynucleotide segment which may be based on a complete or partial naturally occurring nucleotide sequence encoding the polypeptide of interest. The construct may optionally contain other polynucleotide segments. In a similar way, the term “amino acids which can be encoded by polynucleotide constructs” covers amino acids which can be encoded by the polynucleotide constructs defined above, i.e. amino acids such as Ala, Val, Leu, lie, Met, Phe, Trp, Pro, Gly, Ser, Thr, Cys, Tyr, Asn, Glu, Lys, Arg, His, Asp and Gln.

In a further aspect, the invention provides a recombinant vector comprising the polynucleotide construct encoding a Factor VII polypeptide of the invention.

The term “vector”, as used herein, means any nucleic acid entity capable of the amplification in a host cell. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contains a replication origin and at least one selectable gene, i.e., a gene which encodes a product which is readily detectable or the presence of which is essential for cell growth.

In a further aspect, the invention provides a recombinant host cell comprising the polynucleotide construct or the vector. In one embodiment, the host cell is a eukaryotic cell.

In one embodiment, the host cell is of mammalian origin. In one embodiment, the host cell is an insect cell. In one embodiment, the host cell is selected from the group consisting of CHO cells, HEK cells and BHK cells. In one embodiment, the host cell is a microorganism. In one embodiment, the host cell is selected from the group consisting of Escherichia coli, Bacillus subtilis, Rhodococcus, Streptomycetes, Actinomycetes, Corynebacteria, Pseudomonas, Erwinia, Salmonella, Saccharomyces cerevisiae, Picchia, protozoans, Penicillium, Fusarium, Aspergillus, Podospora, and Neurospora.

The term “a host cell”, as used herein, represent any cell, including hybrid cells, in which heterologous DNA can be expressed. Typical host cells includes, but are not limited to bacterial cells, insect cells, yeast cells, mammalian cells, including human cells, such as BHK, CHO, HEK, and COS cells.

In a further aspect, the invention provides a transgenic animal containing and ex-pressing the polynucleotide construct.

In a further aspect, the invention provides a transgenic plant containing and ex-pressing the polynucleotide construct.

In a further aspect the present invention relates to a method for producing a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues, the method comprising cultivating a host cell comprising the polynucleotide construct encoding the Factor VII polypeptide in an appropriate growth medium under conditions allowing expression of the polynucleotide construct and recovering the resulting polypeptide from the growth medium or the host cell. In one embodiment the resulting polypeptide is recovered for the host cell by cell lysis.

As used herein the term “appropriate growth medium” means a medium containing nutrients and other components required for the growth of cells and the expression of the nucleic acid sequence encoding the Factor VII polypeptide of the invention.

In a further aspect, the invention relates to a method for producing the Factor VII polypeptide, the method comprising recovering the polypeptide from milk produced by the transgenic animal.

In a further aspect, the invention relates to a method for producing the Factor VII polypeptide, the method comprising cultivating a host cell of a transgenic plant comprising the polynucleotide construct, and recovering the polypeptide from the resulting plant.

In a further aspect, the invention relates to a pharmaceutical composition comprising a FVII polypeptide, wherein the FVII polypeptide is essentially free of GLA residues; and, optionally, a pharmaceutically acceptable carrier. In one embodiment the FVII polypeptide is wild type human FVIIa.

In a further aspect the present invention relates to a pharmaceutical composition comprising a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues; and, optionally, a pharmaceutically acceptable carrier.

In a further aspect the present invention relates to the use of a FVII polypeptide, wherein the FVII polypeptide is essentially free of GLA residues; for the preparation of a medicament for the treatment of bleeding disorders or bleeding episodes or for the enhancement of the normal haemostatic system. In one embodiment the FVII polypeptide is wild type human FVIIa.

In a further aspect the present invention relates to the use of a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues; for the preparation of a medicament for the treatment of bleeding disorders or bleeding episodes or for the enhancement of the normal haemostatic system. In one embodiment the use is for the treatment of haemophilia A or B.

In the present context, the term “treatment” is means both prevention of an expected bleeding, such as in surgery, and regulation of an already occurring bleeding, such as in trauma, with the purpose of inhibiting or minimizing the bleeding. Prophylactic administration of the Factor VIIa polypeptide according to the invention is thus included in the term “treatment”.

The term “bleeding episodes” is meant to include uncontrolled and excessive bleeding. Bleeding episodes may be a major problem both in connection with surgery and other forms of tissue damage. Uncontrolled and excessive bleeding may occur in subjects having a normal coagulation system and subjects having coagulation or bleeding disorders. As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation Factors XI or VII), clotting factor inhibitors, defective platelet function, thrombocytopenia or von Willebrand's disease.

Excessive bleedings also occur in subjects with a normally functioning blood clotting cascade (no clotting factor deficiencies or -inhibitors against any of the coagulation factors) and may be caused by a defective platelet function, thrombocytopenia or von Willebrand's disease. In such cases, the bleedings may be likened to those bleedings caused by haemophilia because the haemostatic system, as in haemophilia, lacks or has abnormal essential clotting “compounds” (such as platelets or von Willebrand factor protein) that causes major bleedings. In subjects who experience extensive tissue damage in association with surgery or vast trauma, the normal haemostatic mechanism may be overwhelmed by the demand of immediate haemostasis and they may develop bleeding in spite of a normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors. In one embodiment of the invention, the bleeding is associated with haemophilia. In another embodiment, the bleeding is associated with haemophilia with acquired inhibitors. In another embodiment, the bleeding is associated with thrombocytopenia. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with haemorrhagic gastritis. In another embodiment, the bleeding is profuse uterine bleeding. In another embodiment, the bleeding is occurring in organs with a limited possibility for mechanical haemostasis. In another embodiment, the bleeding is occurring in the brain, inner ear region or eyes. In another embodiment, the bleeding is associated with the process of taking biopsies. In another embodiment, the bleeding is associated with anticoagulant therapy.

The term “subject” as used herein is intended to mean any animal, in particular mammals, such as humans, and may, where appropriate, be used interchangeably with the term “patient”.

The term “enhancement of the normal haemostatic system” means an enhancement of the ability to generate thrombin.

In a further aspect the present invention relates to a method for the treatment of bleeding disorders or bleeding episodes in a subject or for the enhancement of the normal haemostatic system, the method comprising administering a therapeutically or prophylactically effective amount of a FVII polypeptide, wherein said FVII polypeptide is essentially free of GLA residues; to a subject in need thereof. In one embodiment the FVII polypeptide is wild type human FVIIa.

In a further aspect the present invention relates to a method for the treatment of bleeding disorders or bleeding episodes in a subject or for the enhancement of the normal haemostatic system, the method comprising administering a therapeutically or prophylactically effective amount of a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues; to a subject in need thereof. In one embodiment the bleeding disorder is haemophilia A or B.

In a further aspect the present invention relates to a Factor VII polypeptide essentially free of GLA residues for use as a medicament.

In a further aspect the present invention relates to a FVII polypeptide comprising one or more amino acid substitutions relative to the amino acid sequence of SEQ ID NO:1, wherein the amino acid substitutions are replacement with any other amino acid of one or more amino acids selected from the group consisting of K157, V158, E296, M298, L305, M306, D309, S314, D334, S336, K337, F374, and wherein the FVII polypeptide is essentially free of GLA residues, for use as a medicament.

### EXEMPLARY ASPECTS AND FEATURES OF THE INVENTION

To better illustrate the invention described herein, a nonlimiting list of exemplary aspects and features of the invention is provided here:

1. A FVII polypeptide comprising one or more amino acid substitutions relative to the amino acid sequence of SEQ ID NO:1, wherein said amino acid substitutions are replacement with any other amino acid of one or more amino acids selected from the group consisting of K157, V158, E296, M298, L305, M306, D309, S314, D334, S336, K337, F374, and wherein said FVII polypeptide is essentially free of GLA residues.

2. The Factor VII polypeptide according to aspect 1, wherein K157 is replaced with any other amino acid.

3. The Factor VII polypeptide according to aspects 1 or 2, wherein V158 is replaced with any other amino acid.

4. The Factor VII polypeptide according to any of aspects 1-3, wherein E296 is replaced with any other amino acid.

5. The Factor VII polypeptide according to any of aspects 1-4, wherein M298 is replaced with any other amino acid.

6. The Factor VII polypeptide according to any of aspects 1-5, wherein L305 is replaced with any other amino acid.

7. The Factor VII polypeptide according to any of aspects 1-6, wherein M306 is replaced with any other amino acid.

8. The Factor VII polypeptide according to any of aspects 1-7, wherein D309 is replaced with any other amino acid.

9. The Factor VII polypeptide according to any of aspects 1-8, wherein S314 is replaced with any other amino acid.

10. The Factor VII polypeptide according to any of aspects 1-9, wherein D334 is replaced with any other amino acid.

11. The Factor VII polypeptide according to any of aspects 1-10, wherein S336 is replaced with any other amino acid.

12. The Factor VII polypeptide according to any of aspects 1-11, wherein K337 is replaced with any other amino acid.

13. The Factor VII polypeptide according to any of aspects 1-12, wherein F374 is replaced with any other amino acid.

14. The Factor VII polypeptide according to any of aspects 1-13, wherein at least one amino acid in the remaining positions in the protease domain has been replaced with any other amino acid.

15. The Factor VII polypeptide according to aspects 14, wherein at the most 20 additional amino acids in the remaining positions in the protease domain have been replaced with any other amino acids.

16. The Factor VII polypeptide according to any of aspects 14-15, wherein at least one amino acid corresponding to an amino acid at a position selected from 159-170 of SEQ ID NO:1 has been replaced with any other amino acid.

17. The Factor VII polypeptide according to any of aspects 14-16, wherein at least one amino acid corresponding to an amino acid at a position selected from 290-304 of SEQ ID NO:1 has been replaced with any other amino acid.

18. The Factor VII polypeptide according to aspect 17, wherein R304 has been replaced by an amino acid selected from the group consisting of Tyr, Phe, Leu, and Met.

19. The Factor VII polypeptide according to any of aspects 14-18, wherein at least one amino acid corresponding to an amino acid at a position selected from 307-312 of SEQ ID NO:1 has been replaced with any other amino acid.

20. The Factor VII polypeptide according to any of aspects 14-19, wherein at least one amino acid corresponding to an amino acid at a position selected from 330-339 of SEQ ID NO:1 has been replaced with any other amino acid.

21. The Factor VII polypeptide according to any of aspects 14-20, wherein A274 has been replaced with any other amino acid.

22. The Factor VII polypeptide according to aspect 21, wherein said A274 has been replaced by an amino acid selected from the group consisting of Met, Leu, Lys, and Arg.

23. The Factor VII polypeptide according to any of aspects 1-22, wherein said K157 has been replaced by an amino acid selected from the group consisting of Gly, Val, Ser, Thr, Asn, Gin, Asp, and Glu.

24. The Factor VII polypeptide according to any of aspects 1-23, wherein said V158 has been replaced by an amino acid selected from the group consisting of Ser, Thr, Asn, Gin, Asp, and Glu.

25. The Factor VII polypeptide according to any of aspects 1-24, wherein said E296 has been replaced by an amino acid selected from the group consisting of Arg, Lys, Ile, Leu and Val.

26. The Factor VII polypeptide according to any of aspects 1-25, wherein said M298 has been replaced by an amino acid selected from the group consisting of Lys, Arg, Gin, and Asn.

27. The Factor VII polypeptide according to any of aspects 1-26, wherein said L305 has been replaced by an amino acid selected from the group consisting of Val, Tyr and Ile.

28. The Factor VII polypeptide according to any of aspects 1-27, wherein M306 has been replaced by an amino acid selected from the group consisting of Asp, and Asn.

29. The Factor VII polypeptide according to any of aspects 1-28, wherein D309 has been replaced by an amino acid selected from the group consisting of Ser, and Thr.

30. The Factor VII polypeptide according to any of aspects 1-29, wherein said S314 has been replaced by an amino acid selected from the group consisting of Gly, Lys, Gin and Glu.

31. The Factor VII polypeptide according to any of aspects 1-30, wherein said D334 has been replaced by an amino acid selected from the group consisting of Gly, and Glu.

32. The Factor VII polypeptide according to any of aspects 1-31, wherein said S336 has been replaced by an amino acid selected from the group consisting of Gly, and Glu.

33. The Factor VII polypeptide according to any of aspects 1-32, wherein said K337 has been replaced by an amino acid selected from the group consisting of Ala, Gly, Val, Ser, Thr, Asn, Gin, Asp, and Glu.

34. The Factor VII polypeptide according to any of aspects 1-33, wherein said F374 has been replaced by an amino acid selected from the group consisting of Pro, and Tyr.

35. The Factor VII polypeptide according to aspect 34, wherein said F374 has been replaced by Tyr.

36. The Factor VII polypeptide according to any of aspects 1-35, wherein said Factor VII polypeptide does not contain the amino acid sequence 1-38 relative to the amino acid sequence of SEQ ID NO:1.

37. The Factor VII polypeptide according to any of aspects 1-35, wherein said Factor VII polypeptide does not contain the amino acid sequence 1-44 relative to the amino acid sequence of SEQ ID NO:1.

38. The Factor VII polypeptide according to any of aspects 1-35, wherein said Factor VII polypeptide does not contain the amino acid sequence 1-152 relative to the amino acid sequence of SEQ ID NO:1.

39. The Factor VII polypeptide according to any of aspects 1-36, wherein said Factor VII polypeptide at least consists of the amino acid sequence 39-406 relative to the amino acid sequence of SEQ ID NO:1.

40. The Factor VII polypeptide according to any of aspects 1-37, wherein said Factor VII polypeptide at least consists of the amino acid sequence 45-406 relative to the amino acid sequence of SEQ ID NO:1.

41. The Factor VII polypeptide according to any of aspects 1-38, wherein said Factor VII polypeptide at least consists of the amino acid sequence 153-406 relative to the amino acid sequence of SEQ ID NO:1.

42. The Factor VII polypeptide according to any of aspects 1-41, wherein the amino acid has been replaced with any other amino acid which can be encoded by polynucleotide constructs.

43. The Factor VII polypeptide according to any of aspects 1-42, wherein said Factor VII polypeptide is in activated form.

44. The Factor VII polypeptide according to any of aspects 1-43, wherein the ratio between the proteolytic activity of said Factor VII polypeptide and the proteolytic activity of the native Factor Vlla polypeptide shown in SEQ ID NO:1 is at least about 1.25.

45. The Factor VII polypeptide according to aspect 44, wherein said ratio is at least about 2.0, preferably at least about 4.0.

46. A polynucleotide construct encoding a Factor VII polypeptide according to any of aspects 1-45.

47. The polynucleotide construct according to aspect 46, which is a vector.

48. A host cell comprising the polynucleotide construct according to any one of the aspects 46-47.

49. The host cell according to aspect 48, which is a eukaryotic cell.

50. The host cell according to aspect 49, which is of mammalian origin.

51. The host cell according to aspect 49, which is an insect cell.

52. The host cell according to aspect 50, wherein the cell is selected from the group consisting of CHO cells, HEK cells and BHK cells.

53. The host cell according to aspect 48, which is a microorganism.

54. The host cell according to aspect 53, wherein the microorganism is selected from the group consisting of Escherichia coli, Bacillus subtilis, Rhodococcus, Streptomycetes, Actinomycetes, Corynebacteria, Pseudomonas, Erwinia, Salmonella, Saccharomyces cerevisiae, Picchia, protozoans, Penicillium, Fusarium, Aspergillus, Podospora, and Neurospora.

55. A transgenic animal containing and expressing the polynucleotide construct as defined in aspect 46.

56. A transgenic plant containing and expressing the polynucleotide construct as defined in aspect 46.

57. A method for producing the Factor VII polypeptide defined in any of aspects 1-45, the method comprising cultivating a host cell as defined in any one of aspects 48-54 in an appropriate growth medium under conditions allowing expression of the polynucleotide construct and recovering the resulting polypeptide from said growth medium or said host cell.

58. A method for producing the Factor VII polypeptide defined in any of aspects 1-45, the method comprising recovering the Factor VII polypeptide from milk produced by the transgenic animal defined in aspect 55.

59. A method for producing the Factor VII polypeptide defined in any of aspects 1-45, the method comprising cultivating a host cell of a transgenic plant as defined in aspect 56, and recovering the Factor VII polypeptide from the plant.

60. A pharmaceutical composition comprising a FVII polypeptide, wherein said FVII polypeptide is essentially free of GLA residues; and, optionally, a pharmaceutically acceptable carrier.

61. A pharmaceutical composition comprising a FVII polypeptide comprising one or more amino acid substitutions relative to the amino acid sequence of SEQ ID NO:1, wherein said amino acid substitutions are replacement with any other amino acid of one or more amino acids selected from the group consisting of K157, V158, E296, M298, L305, M306, D309, S314, D334, S336, K337, F374, and wherein said FVII polypeptide is essentially free of GLA residues; and, optionally, a pharmaceutically acceptable carrier.

62. The pharmaceutical composition according to aspect 60, wherein said Factor VII polypeptide is wild type human FVII essentially free of GLA residues; and, optionally, a pharmaceutically acceptable carrier.

63. The pharmaceutical composition according to any of aspects 60-62, wherein said Factor VII polypeptide does not contain the amino acid sequence 1-38 relative to the amino acid sequence of SEQ ID NO:1.

64. The pharmaceutical composition according to any of aspects 60-63, wherein said Factor VII polypeptide does not contain the amino acid sequence 1-44 relative to the amino acid sequence of SEQ ID NO:1.

65. The pharmaceutical composition according to any of aspects 60-64, wherein said Factor VII polypeptide does not contain the amino acid sequence 1-152 relative to the amino acid sequence of SEQ ID NO:1.

66. The pharmaceutical composition according to any of aspects 60, 62-65, wherein said Factor VII polypeptide comprises the amino acid sequence 39-406 of SEQ ID NO:1.

67. The pharmaceutical composition according to any of aspects 60, 62-66, wherein said Factor VII polypeptide comprises the amino acid sequence 45-406 of SEQ ID NO:1.

68. The pharmaceutical composition according to any of aspects 60, 62-67, wherein said Factor VII polypeptide comprises the amino acid sequence 153-406 of SEQ ID NO:1.

69. The pharmaceutical composition according to any of aspects 60-68, wherein the amino acid has been replaced with any other amino acid which can be encoded by polynucleotide constructs.

70. The pharmaceutical composition according to any of aspects 60-69, wherein said Factor VII polypeptide is in activated form.

71. A pharmaceutical composition comprising a Factor VII polypeptide as defined in any of aspects 1-45, and, optionally, a pharmaceutically acceptable carrier.

72. Use of a FVII polypeptide, wherein said FVII polypeptide is essentially free of GLA residues; for the preparation of a medicament for the treatment of bleeding disorders or bleeding episodes or for the enhancement of the normal haemostatic system.

73. Use of a FVII polypeptide comprising one or more amino acid substitutions relative to the amino acid sequence of SEQ ID NO:1, wherein said amino acid substitutions are replacement with any other amino acid of one or more amino acids selected from the group consisting of K157, V158, E296, M298, L305, M306, D309, S314, D334, S336, K337, F374, and wherein said FVII polypeptide is essentially free of GLA residues; for the preparation of a medicament for the treatment of bleeding disorders or bleeding episodes or for the enhancement of the normal haemostatic system.

74. Use according to aspect 72, wherein said Factor VII polypeptide is wild type human FVII essentially free of GLA residues; and, optionally, a pharmaceutically acceptable carrier.

75. Use according to any of aspects 72-74, wherein said Factor VII polypeptide does not contain the amino acid sequence 1-38 relative to the amino acid sequence of SEQ ID NO:1.

76. Use according to any of aspects 72-75, wherein said Factor VII polypeptide does not contain the amino acid sequence 1-44 relative to the amino acid sequence of SEQ ID NO:1.

77. Use according to any of aspects 72-76, wherein said Factor VII polypeptide does not contain the amino acid sequence 1-152 relative to the amino acid sequence of SEQ ID NO:1.

78. Use according to any of aspects 72, 74-77, wherein said Factor VII polypeptide comprises the amino acid sequence 39-406 of SEQ ID NO:1.

79. Use according to any of aspects 72, 74-78, wherein said Factor VII polypeptide comprises the amino acid sequence 45-406 of SEQ ID NO:1.

80. Use according to any of aspects 72, 74-79, wherein said Factor VII polypeptide comprises the amino acid sequence 153-406 of SEQ ID NO:1.

81. Use according to any of aspects 72-80, wherein the amino acid has been replaced with any other amino acid which can be encoded by polynucleotide constructs.

82. Use according to any of aspects 72-81, wherein said Factor VII polypeptide is in activated form.

83. Use of a Factor VII polypeptide as defined in any of aspects 1-45 for the preparation of a medicament for the treatment of bleeding disorders or bleeding episodes or for the enhancement of the normal haemostatic system.

84. Use according to any of aspects 72-83 for the treatment of haemophilia A or B.

85. A method for the treatment of bleeding disorders or bleeding episodes in a subject or for the enhancement of the normal haemostatic system, the method comprising administering a therapeutically or prophylactically effective amount of a FVII polypeptide, wherein said FVII polypeptide is essentially free of GLA residues; to a subject in need thereof.

86. A method for the treatment of bleeding disorders or bleeding episodes in a subject or for the enhancement of the normal haemostatic system, the method comprising administering a therapeutically or prophylactically effective amount of a FVII polypeptide comprising one or more amino acid substitutions relative to the amino acid sequence of SEQ ID NO:1, wherein said amino acid substitutions are replacement with any other amino acid of one or more amino acids selected from the group consisting of K157, V158, E296, M298, L305, M306, D309, S314, D334, S336, K337, F374, and wherein said FVII polypeptide is essentially free of GLA residues; to a subject in need thereof.

87. The method according to aspect 85, wherein said Factor VII polypeptide is wild type human FVII essentially free of GLA residues; and, optionally, a pharmaceutically acceptable carrier.

88. The method according to any of aspects 85-87, wherein said Factor VII polypeptide does not contain the amino acid sequence 1-38 relative to the amino acid sequence of SEQ ID NO: 1.

89. The method according to any of aspects 85-88, wherein said Factor VII polypeptide does not contain the amino acid sequence 1-44 relative to the amino acid sequence of SEQ ID NO:1.

90. The method according to any of aspects 85-89, wherein said Factor VII polypeptide does not contain the amino acid sequence 1-152 relative to the amino acid sequence of SEQ ID NO:1.

91. The method according to any of aspects 85, 87-90, wherein said Factor VII polypeptide comprises the amino acid sequence 39-406 of SEQ ID NO:1.

92. The method according to any of aspects 85, 87-91, wherein said Factor VII polypeptide comprises the amino acid sequence 45-406 of SEQ ID NO:1.

93. The method according to any of aspects 85, 87-92, wherein said Factor VII polypeptide comprises the amino acid sequence 153-406 of SEQ ID NO:1.

94. The method according to any of aspects 85-93, wherein the amino acid has been replaced with any other amino acid which can be encoded by polynucleotide constructs.

95. The method according to any of aspects 85-94, wherein said Factor VII polypeptide is in activated form.

96. A method for the treatment of bleeding disorders or bleeding episodes in a subject or for the enhancement of the normal haemostatic system, the method comprising administering a therapeutically or prophylactically effective amount of a Factor VII polypeptide as defined in any of aspects 1-45 to a subject in need thereof.

97. A Factor VII polypeptide essentially free of GLA residues for use as a medicament.

98. The Factor VII polypeptide according to aspect 97, wherein said Factor VII polypeptide is wild type human FVII essentially free of GLA residues; and, optionally, a pharmaceutically acceptable carrier.

99. The Factor VII polypeptide according to any of aspects 97-98, wherein said Factor VII polypeptide does not contain the amino acid sequence 1-38 relative to the amino acid sequence of SEQ ID NO:1.

100. The Factor VII polypeptide according to any of aspects 97-99, wherein said Factor VII polypeptide does not contain the amino acid sequence 1-44 relative to the amino acid sequence of SEQ ID NO:1.

101. The Factor VII polypeptide according to any of aspects 97-100, wherein said Factor VII polypeptide does not contain the amino acid sequence 1-152 relative to the amino acid sequence of SEQ ID NO:1.

102. The Factor VII polypeptide according to any of aspects 97-101, wherein said Factor VII polypeptide comprises the amino acid sequence 39-406 of SEQ ID NO:1.

103. The Factor VII polypeptide according to any of aspects 97-102, wherein said Factor VII polypeptide comprises the amino acid sequence 45-406 of SEQ ID NO:1.

104. The Factor VII polypeptide according to any of aspects 97-103, wherein said Factor VII polypeptide comprises the amino acid sequence 153-406 of SEQ ID NO: 1.

105. The Factor VII polypeptide according to any of aspects 97-104, wherein the amino acid has been replaced with any other amino acid which can be encoded by polynucleotide constructs.

106. The Factor VII polypeptide according to any of aspects 97-105, wherein said Factor VII polypeptide is in activated form.

107. A Factor VII polypeptide as defined in any of aspects 1-45 for use as a medicament.

These aspects are more fully described in, and additional aspects, features, and advantages of the invention will be apparent from, the description of the invention provided herein.

### DETAILED DESCRIPTION OF THE INVENTION

The inventors of the present invention have realized that it will be feasible to prepare a Factor VII polypeptide having substantially the same or increased proteolytic activity compared to recombinant wild type human Factor VIIa, where the gamma-carboxyglutamic acid containing lipid membrane binding domain is lacking or is non-functional.

Recent advances in our understanding of the mechanisms regulating the activity of FVIIa have pinpointed side chains that function as zymogenicity determinants in the free enzyme. Replacements of these amino acid residues have resulted in FVIIa molecules with increased intrinsic (TF-independent) catalytic efficiency. The relatively high intrinsic activity of some of these FVIIa variants suggests that the zymogen-like conformation of free factor VIIa is dictated by a limited number of key amino acid residues. One of these superactive FVII a variants, containing the mutations at positions 158, 296 and 298, exhibits several properties resembling TF-bound rather than free FVIIa. Apart from increased intrinsic enzymatic activity and inhibitor susceptibility as compared with wild-type FVIIa, these FVIIa variants have a diminished requirement for calcium ions and a more deeply buried protease domain N-terminus (Ile153) indicating salt bridge formation of this residue with Asp343.

As already suggested wild-type human FVIIa and FVIIa variants with increased proteolytic activity as compared to wild-type human FVIIa have different properties. Whereas free wild-type human FVIIa has a zymogen-like conformation, FVIIa variants with increased proteolytic activity as compared to wild-type human FVIIa exhibits several properties resembling TF-bound FVIIa.

According to the present invention, a FVII polypeptide which is “essentially free of GLA residues” is a FVII polypeptide where there is substantially no host-cell derived GLA residues, i.e. 4-carboxyglutamic acid (gamma-carboxyglutamic acid).

One aspect of the present invention relates to a FVII polypeptide essentially free of GLA residues. In one embodiment the FVII polypeptide is not a FVII polypeptide consisting of 39-406 relative to the amino acid sequence of SEQ ID NO:1 (des(1-38)-recombinant human FVIIa). In one embodiment the FVII polypeptide is not a FVII polypeptide consisting of 45-406 relative to the amino acid sequence of SEQ ID NO:1 (des(1-44)-recombinant human FVIIa).

In one embodiment the FVII polypeptide has no GLA residues at all. In one embodiment of the invention, the GLA residues at any position selected from the list consisting of 6, 7, 14, 16, 19, 20, 25, 26, 29, and 35 relative to the amino acid sequence of SEQ ID NO:1 has been replaced by any other amino acid. In one embodiment of the invention, the GLA residues at any position selected from the list consisting of 6, 7, 14, 16, 19, 20, 25, 26, 29, and 35 relative to the amino acid sequence of SEQ ID NO:1 has been replaced by an amino acid selected from the group consisting of Glu, Gln, Asp, and Asn. In one embodiment of the invention, the GLA residues at any position selected from 6, 7, 14, 16, 19, 20, 25, 26, 29, and 35 relative to the amino acid sequence of SEQ ID NO:1 has been replaced by a Glu. In one embodiment of the invention, the GLA residues at any position selected from 6, 7, 14, 16, 19, 20, 25, 26, 29, and 35 relative to the amino acid sequence of SEQ ID NO:1 has been replaced by a Gin. It is to be understood, that the replacement of GLA residues at any position selected from 6, 7, 14, 16, 19, 20, 25, 26, 29, and 35 relative to the amino acid sequence of SEQ ID NO:1 may be replaced by Glu by the removal of a functional propeptide from a nucleotide construct encoding the FVII polypeptide. Thus, in one embodiment of the invention, the polynucleotide construct encoding a Factor VII polypeptide according to the invention is devoid of a functional propeptide.

In one embodiment of the invention 1, 2, 3, 4, 5, 6, 7, 8, 9, or 10 GLA residues at any position selected from the list consisting of 6, 7, 14, 16, 19, 20, 25, 26, 29, and 35 relative to the amino acid sequence of SEQ ID NO:1 has been replaced by any other amino acid.

In another embodiment of the invention, the FVII polypeptide is essentially free of GLA residues by synthesis in a host cell, which is not able to gamma-carboxylate. In one embodiment, the host cell does not express a gamma-carboxylase.

In another embodiment of the invention, the FVII polypeptide is essentially free of GLA residues by synthesis in a host cell in the presence of a vitamin-K antagonist. In one embodiment, the vitamin K antagonist is selected from the list consisting of warfarin, coumarin, and acenocoumarol.

According to the present invention, a FVII polypeptide may have a lipid membrane binding domain not normally present in human wild type FVII and/or derived from another polypeptide. Suitable lipid membrane binding domains are disclosed in U.S. Pat. No. 5,225,537, which is hereby incorporated by reference in its entirety.

In one embodiment of the invention, the factor VII polypeptide is a polypeptide, wherein K157 is replaced with any other amino acid.

The term “any other amino acid” as used herein means one amino acid that are different from that amino acid naturally present at that position. This includes but is not limited to amino acids that can be encoded by a polynucleotide. Preferably the different amino acid is in natural L-form and can be encoded by a polynucleotide. A specific example being L-cysteine (Cys).

In one embodiment of the invention, the factor VII polypeptide is a polypeptide, wherein V158 is replaced with any other amino acid.

In one embodiment of the invention, the factor VII polypeptide is a polypeptide, wherein E296 is replaced with any other amino acid.

In one embodiment of the invention, the factor VII polypeptide is a polypeptide, wherein M298 is replaced with any other amino acid.

In one embodiment of the invention, the factor VII polypeptide is a polypeptide, wherein L305 is replaced with any other amino acid.

In one embodiment of the invention, the factor VII polypeptide is a polypeptide, wherein M306 is replaced with any other amino acid.

In one embodiment of the invention, the factor VII polypeptide is a polypeptide, wherein D309 is replaced with any other amino acid.

In one embodiment of the invention, the factor VII polypeptide is a polypeptide, wherein S314 is replaced with any other amino acid.

In one embodiment of the invention, the factor VII polypeptide is a polypeptide, wherein D334 is replaced with any other amino acid.

In one embodiment of the invention, the factor VII polypeptide is a polypeptide, wherein S336 is replaced with any other amino acid.

In one embodiment of the invention, the factor VII polypeptide is a polypeptide, wherein K337 is replaced with any other amino acid.

In one embodiment of the invention, the factor VII polypeptide is a polypeptide, wherein F374 is replaced with any other amino acid.

In one embodiment of the invention, the factor VII polypeptide is a polypeptide, wherein at least one amino acid in the remaining positions in the protease domain has been replaced with any other amino acid.

In one embodiment of the invention, the factor VII polypeptide is a polypeptide, wherein at the most 20 additional amino acids in the remaining positions in the protease domain have been replaced with any other amino acids.

In one embodiment of the invention, the factor VII polypeptide is a polypeptide, wherein at least one amino acid corresponding to an amino acid at a position selected from 159-170 of SEQ ID NO:1 has been replaced with any other amino acid.

In one embodiment of the invention, the factor VII polypeptide is a polypeptide, wherein at least one amino acid corresponding to an amino acid at a position selected from 290-304 of SEQ ID NO:1 has been replaced with any other amino acid.

In one embodiment of the invention, the factor VII polypeptide is a polypeptide, wherein R304 has been replaced by an amino acid selected from the group consisting of Tyr, Phe, Leu, and Met.

In one embodiment of the invention, the factor VII polypeptide is a polypeptide, wherein at least one amino acid corresponding to an amino acid at a position selected from 307-312 of SEQ ID NO:1 has been replaced with any other amino acid.

In one embodiment of the invention, the factor VII polypeptide is a polypeptide, wherein at least one amino acid corresponding to an amino acid at a position selected from 330-339 of SEQ ID NO:1 has been replaced with any other amino acid.

In one embodiment of the invention, the factor VII polypeptide is a polypeptide, wherein A274 has been replaced with any other amino acid.

In one embodiment of the invention, the factor VII polypeptide is a polypeptide, wherein the A274 has been replaced by an amino acid selected from the group consisting of Met, Leu, Lys, and Arg.

In one embodiment of the invention, the factor VII polypeptide is a polypeptide, wherein the K157 has been replaced by an amino acid selected from the group consisting of Gly, Val, Ser, Thr, Asn, Gln, Asp, and Glu.

In one embodiment of the invention, the factor VII polypeptide is a polypeptide, wherein the V158 has been replaced by an amino acid selected from the group consisting of Ser, Thr, Asn, Gln, Asp, and Glu.

In one embodiment of the invention, the factor VII polypeptide is a polypeptide, wherein the E296 has been replaced by an amino acid selected from the group consisting of Arg, Lys, Ile, Leu and Val.

In one embodiment of the invention, the factor VII polypeptide is a polypeptide, wherein the M298 has been replaced by an amino acid selected from the group consisting of Lys, Arg, Gln, and Asn.

In one embodiment of the invention, the factor VII polypeptide is a polypeptide, wherein the L305 has been replaced by an amino acid selected from the group consisting of Val, Tyr and Ile.

In one embodiment of the invention, the factor VII polypeptide is a polypeptide, wherein M306 has been replaced by an amino acid selected from the group consisting of Asp, and Asn.

In one embodiment of the invention, the factor VII polypeptide is a polypeptide, wherein D309 has been replaced by an amino acid selected from the group consisting of Ser, and Thr.

In one embodiment of the invention, the factor VII polypeptide is a polypeptide, wherein the S314 has been replaced by an amino acid selected from the group consisting of Gly, Lys, Gln and Glu.

In one embodiment of the invention, the factor VII polypeptide is a polypeptide, wherein the D334 has been replaced by an amino acid selected from the group consisting of Gly, and Glu.

In one embodiment of the invention, the factor VII polypeptide is a polypeptide, wherein the S336 has been replaced by an amino acid selected from the group consisting of Gly, and Glu.

In one embodiment of the invention, the factor VII polypeptide is a polypeptide, wherein the K337 has been replaced by an amino acid selected from the group consisting of Ala, Gly, Val, Ser, Thr, Asn, Gin, Asp, and Glu.

In one embodiment of the invention, the factor VII polypeptide is a polypeptide, wherein the F374 has been replaced by an amino acid selected from the group consisting of Pro, and Tyr.

In one embodiment of the invention, the factor VII polypeptide is a polypeptide, wherein the F374 has been replaced by Tyr.

In one embodiment of the invention, the factor VII polypeptide is a polypeptide, wherein the Factor VII polypeptide does not contain the amino acid sequence 1-38 relative to the amino acid sequence of SEQ ID NO:1. In one embodiment, the amino acid sequence 1-38 relative to the amino acid sequence of SEQ ID NO:1 has been removed by a proteolytic enzyme after synthesis of the factor VII polypeptide. In one embodiment, the polynucleotide sequence encoding the amino acid sequence 1-38 relative to the amino acid sequence of SEQ ID NO:1 has been removed from the polynucleotide construct encoding the factor VII polypeptide.

In one embodiment of the invention, the factor VII polypeptide is a polypeptide, wherein the Factor VII polypeptide does not contain the amino acid sequence 1-44 relative to the amino acid sequence of SEQ ID NO:1. In one embodiment, the amino acid sequence 1-44 relative to the amino acid sequence of SEQ ID NO:1 has been removed by a proteolytic enzyme after synthesis of the factor VII polypeptide. In one embodiment, the polynucleotide sequence encoding the amino acid sequence 1-44 relative to the amino acid sequence of SEQ ID NO:1 has been removed from the polynucleotide construct encoding the factor VII polypeptide.

In one embodiment of the invention, the factor VII polypeptide is a polypeptide, wherein the Factor VII polypeptide does not contain the amino acid sequence 1-85 relative to the amino acid sequence of SEQ ID NO:1.

In one embodiment of the invention, the factor VII polypeptide is a polypeptide, wherein the Factor VII polypeptide does not contain the amino acid sequence 1-90 relative to the amino acid sequence of SEQ ID NO:1.

In one embodiment of the invention, the factor VII polypeptide is a polypeptide, wherein the Factor VII polypeptide does not contain the amino acid sequence 1-152 relative to the amino acid sequence of SEQ ID NO:1.

In one embodiment of the invention, the factor VII polypeptide is a polypeptide, wherein the Factor VII polypeptide at least consists of the amino acid sequence 39-406 relative to the amino acid sequence of SEQ ID NO:1.

In one embodiment of the invention, the factor VII polypeptide is a polypeptide, wherein the Factor VII polypeptide at least consists of the amino acid sequence 45-406 relative to the amino acid sequence of SEQ ID NO:1.

In one embodiment of the invention, the factor VII polypeptide is a polypeptide, wherein the Factor VII polypeptide at least consists of the amino acid sequence 86-406 relative to the amino acid sequence of SEQ ID NO:1.

In one embodiment of the invention, the factor VII polypeptide is a polypeptide, wherein the Factor VII polypeptide at least consists of the amino acid sequence 91-406 relative to the amino acid sequence of SEQ ID NO:1.

In one embodiment of the invention, the factor VII polypeptide is a polypeptide, wherein the Factor VII polypeptide at least consists of the amino acid sequence 153-406 relative to the amino acid sequence of SEQ ID NO:1.

In one embodiment of the invention, the factor VII polypeptide is a polypeptide, wherein the amino acid has been replaced with any other amino acid which can be encoded by polynucleotide constructs.

In one embodiment of the invention, the factor VII polypeptide is a polypeptide, wherein the Factor VII polypeptide is in activated form.

In one embodiment of the invention, the factor VII polypeptide is a polypeptide, wherein the ratio between the proteolytic activity of the Factor VII polypeptide and the proteolytic activity of the native Factor VIIa polypeptide shown in SEQ ID NO:1 is at least about 1.25. In one embodiment of the invention, the factor VII polypeptide is a polypeptide, wherein the ratio is at least about 2.0, preferably at least about 4.0.

In a further embodiment of the invention, the Factor VII polypeptide has a proteolytic activity higher than wild type human FVIIa.

In a further embodiment of the invention, the Factor VII polypeptide is wild type human FVII, which does not contain the amino acid sequence 1-38 relative to the amino acid sequence of SEQ ID NO:1.

In a further embodiment of the invention, the Factor VII polypeptide is wild type human FVII, which does not contain the amino acid sequence 1-44 relative to the amino acid sequence of SEQ ID NO:1.

In a further embodiment of the invention, the Factor VII polypeptide is wild type human FVII, which does not contain the amino acid sequence 1-152 relative to the amino acid sequence of SEQ ID NO:1.

In a further embodiment of the invention, the Factor VII polypeptide comprises the amino acid sequence 39-406 of SEQ ID NO:1 of wild type human FVII.

In a further embodiment of the invention, the Factor VII polypeptide comprises the amino acid sequence 45-406 of SEQ ID NO:1 of wild type human FVII.

In a further embodiment of the invention, the Factor VII polypeptide comprises the amino acid sequence 153-406 of SEQ ID NO:1 of wild type human FVII.

In a further embodiment of the invention, the Factor VII polypeptide comprises amino acid substitutions relative to the amino acid sequence of SEQ ID NO:1 selected from the group consisting of: L305V, L305V/M306D/D309S, L3051, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/M158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158V/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V1158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V1158T, K316H/L305V/K337A/M298Q, K316H/L305V/K337A/E296V, K316H/L305V/K337A/V1158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V1158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K3160/L305V/K337A/M298Q, K316Q/L305V/K337A/E296V, K316Q/L305V/K337A/V158D, K3160/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K3160/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M2980, F374Y/L305V/V158T, F374Y/L305V/S314E, F374Y/K337A/S314E, F374Y/K337A/V1158T, F374Y/K337A/M298Q, F374Y/K337A/E296V, F374Y/K337A/V158D, F374Y/V158D/S314E, F374Y/V158D/M298Q, F374Y/V158D/E296V, F374Y/V1158T/S314E, F374Y/V158T/M2980, F374Y/V158T/E296V, F374Y/E296V/S314E, F374Y/S314E/M298Q, F374Y/E296V/M298Q, F374Y/L305V/K337A/V 158D, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V1158T/E296V, F374Y/K337A/M298Q/E296V, F374Y/K337A/M298Q/V158D, F374Y/K337A/E296V/V158D, F374Y/V158D/S314E/M298Q, F374Y/V1158D/S314E/E296V, F374Y/V158D/M298Q/E296V, F374Y/V1158T/S314E/E296V, F374Y/V1158T/S314E/M2980, F374Y/V158T/M298Q/E296V, F374Y/E296V/S314E/M298Q, F374Y/L305V/M2980/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M2980/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M2980/K337A/V158T/S314E, F374Y/V158D/E296V/M2980/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E, F374Y/L305V/V158D/E296V/M298Q/K337A/S314E, S52A-Factor VII, S60A-Factor VII; R152E-Factor VII, S344A-Factor VII, and P11Q/K33E, T106N, K143N/N145T, V253N, R290N/A292T, G291N, R315N/V317T, K143N/N145T/R315N/V317T; and FVII having substitutions, additions or deletions in the amino acid sequence from 233Thr to 240Asn, FVII having substitutions, additions or deletions in the amino acid sequence from 304Arg to 329Cys.

In a further embodiment of the invention, the ratio between the proteolytic activity of the Factor VII polypeptide and the proteolytic activity of the wild type human Factor VIIa is at least about 1.25 when tested in assay 1 as described herein.

In a further embodiment of the invention, the ratio between the proteolytic activity of the Factor VII polypeptide and the proteolytic activity of the wild type human Factor VIIa is at least about 2.0, such as at least about 4.0, when tested in assay 1 as described herein.

In a further embodiment of the invention, the ratio between the proteolytic activity of the Factor VII polypeptide and the proteolytic activity of the wild type human Factor VIIa is at least about 1.25 when tested in assay 2 as described herein.

In a further embodiment of the invention, the ratio between the proteolytic activity of the Factor VII polypeptide and the proteolytic activity of the wild type human Factor VIIa is at least about 2.0, such as at least about 4.0, when tested in assay 2 as described herein.

As used herein, “wild type human FVIIa” is a polypeptide having the amino acid sequence disclosed in U.S. Pat. No. 4,784,950 (FIG. 1).

As used herein, the terms “Factor VII polypeptide” or “FVII polypeptide” means any protein comprising the amino acid sequence 1-406 of native human Factor VII (SEQ ID NO: 1) or variants thereof. This includes but is not limited to human Factor VII, human Factor VIIa and variants thereof. This includes variants of Factor VII exhibiting substantially the same or increased proteolytic activity compared to recombinant wild type human Factor VIIa, as well as Factor VII derivatives and Factor VII conjugates. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like.

A “lipid membrane binding domain” is in the present context a protein domain that binds to cell membrane lipid domains. “Lipid membrane binding domains” typically include gamma-carboxyglutamic residues and will normally be derived from a vitamin K dependent protein such as factors VII, IX, and X; protein C, protein S; osteocalcin, matrix Gla protein, and proline-rich Gla protein 1. Other lipid membrane binding domains are disclosed in U.S. Pat. No. 5,225,537, which US patent is hereby incorporated by reference.

The term “N-terminal Gla-domain” or just “Gla-domain” specifically means the amino acid sequence 1-37 of Factor VII set forth in SEQ ID NO: 1.

The three-letter indication “Gla” means 4-carboxyglutamic acid (gamma-carboxyglutamate).

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota).

The term “PEGylated human Factor VIIa” means human Factor VIIa, having a PEG molecule conjugated to a Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

The biological activity of Factor VIIa in blood clotting derives from its ability to (i) bind to tissue factor (TF) and (ii) catalyze the proteolytic cleavage of Factor 1× or Factor X to produce activated Factor 1× or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Alternatively, Factor VIIa biological activity may be quantified by the generation of thrombin on platelets as described in Persson et al, PNAS 98, 13583-13588, 2001.

The term “substantially the same or increased proteolytic activity compared to recombinant wild type human Factor VIIa”, as used herein, means an activity more than 50% of the activity of recombinant wild type human Factor VIIa, when tested in one or more of a clotting assay, or proteolysis assay, as described herein. In one embodiment the activity is more than 80% of the activity of recombinant wild type human Factor VIIa. In another embodiment the activity is more than 90% of the activity of recombinant wild type human Factor VIIa. In a further embodiment the activity is more than 100% of the activity of recombinant wild type human Factor VIIa. In a further embodiment the activity is more than 120% of the activity of recombinant wild type human Factor VIIa. In a further embodiment the activity is more than 200% of the activity of recombinant wild type human Factor VIIa. In a further embodiment the activity is more than 400% of the activity of recombinant wild type human Factor VIIa.

Variants of Factor VII, whether exhibiting substantially the same or increased proteolytic activity compared to recombinant wild type human Factor VIIa, include, without limitation, polypeptides having an amino acid sequence that differs from the sequence of wild-type Factor VII by insertion, deletion, or substitution of one or more amino acids.

The terms “variant” or “variants”, as used herein, is intended to designate Factor VII having the sequence of wild type human FVII, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. The “variant” or “variants” within this definition still have FVII activity in its activated form. In one embodiment the FVII polypeptide variant comprises at least the amino acid sequence 91-368 of native human Factor VII (SEQ ID NO: 1).

In one embodiment a variant is 70% identical with the sequence of wild type human FVII. In one embodiment a variant is 80% identical with the sequence of wild type human FVII. In another embodiment a variant is 90% identical with the sequence of wild type human FVII. In a further embodiment a variant is 95% identical with the sequence of wild type human FVII.

Non-limiting examples of Factor VII variants having substantially the same or increased proteolytic activity compared to recombinant wild type human Factor VIIa include S52A-FVIIa, S60A-FVIIa (Iino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999);. FVII variants as disclosed in PCT/DK02/00189; and FVII variants exhibiting increased proteolytic stability as disclosed in WO 02/38162 (Scripps Research Institute); and FVII variants as disclosed in WO 01/58935 (Maxygen ApS) and WO 04/029091 (Maxygen ApS).

Non-limiting examples of Factor VII variants having substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, U.S. Pat. No. 6,017,882 and U.S. Pat. No. 6,747,003, US patent application 20030100506 (University of Minnesota) and WO 00/66753, US patent applications US 20010018414, US 2004220106, and US 200131005, U.S. Pat. No. 6,762,286 and U.S. Pat. No. 6,693,075 (University of Minnesota); and FVII variants as disclosed in WO 01/58935, US patent U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.).

Examples of variants of factor VII include, without limitation, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M2980-FVII, L305V/V158D/E296V/K337A-FVII, L305V/V158D/E296V/M298Q/K337A-FVII, L305V/V158T/E296V/M2980/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M2980-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M2980-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M2980-FVII, K316Q/L305V/V158T/E296V-FVII, K316Q/L305V/E296V/M298Q-FVII, K316Q/L305V/V158D/E296V/M2980-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M2980-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M2980-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M2980/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V 158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M2980/K337A-FVII, F374Y/V158T/E296V/M298Q/S314E-FVII, F374Y/L305V/V158T/K337A/S314E-FVII, F374Y/V158T/M2980/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153Ile to 223Arg.

The terminology for amino acid substitutions used is as follows. The first letter represents the amino acid naturally present at a position of human wild type FVII. The following number represents the position in human wild type FVII. The second letter represent the different amino acid substituting for (replacing) the natural amino acid. An example is M298Q, where a methionine at position 298 of human wild type FVII is replaced by a glutamine. In another example, V158T/M298Q, the valine in position 158 of human wild type FVII is replaced by a threonine and the methionine in position 298 of human wild type FVII is replaced by a Glutamine in the same Factor VII polypeptide.

In a further embodiment of the invention, the Factor VII polypeptide is a polypeptide, wherein the ratio between the activity of the Factor VII polypeptide and the activity of the wild type human Factor VIIa is at least about 1.25. In one embodiment the ratio between the activity of the Factor VII polypeptide and the activity of the wild type human Factor VIIa is at least about 2.0. In a further embodiment the ratio between the activity of the Factor VII polypeptide and the activity of the wild type human Factor VIIa is at least about 4.0.

In a further embodiment of the invention, the Factor VII polypeptide is a polypeptide, wherein the ratio between the activity of the Factor VII polypeptide and the activity of the wild type human Factor VIIa is at least about 1.25 when tested in a Factor VIIa activity assay. In one embodiment the ratio between the activity of the Factor VII polypeptide and the activity of the wild type human Factor VIIa is at least about 2.0 when tested in a Factor VIIa activity assay. In a further embodiment the ratio between the activity of the Factor VII polypeptide and the activity of the wild type human Factor VIIa is at least about 4.0 when tested in a Factor VIIa activity assay. The Factor VIIa activity may be measured by the assays described under “assays”.

In a further embodiment of the invention, the Factor VII polypeptide is a polypeptide, wherein the ratio between the activity of the Factor VII polypeptide and the activity of the wild type human Factor VIIa is at least about 1.25 when tested in the “In Vitro Hydrolysis Assay”. In one embodiment the ratio between the activity of the Factor VII polypeptide and the activity of the wild type human Factor VIIa is at least about 2.0 when tested in the “In Vitro Hydrolysis Assay”. In a further embodiment the ratio between the activity of the Factor VII polypeptide and the activity of the wild type human Factor VIIa is at least about 4.0 when tested in the “In Vitro Hydrolysis Assay”.

In a further embodiment of the invention, the Factor VII polypeptide is a polypeptide, wherein the ratio between the activity of the Factor VII polypeptide and the activity of the wild type human Factor VIIa is at least about 1.25 when tested in the “In Vitro Proteolysis Assay”. In one embodiment the ratio between the activity of the Factor VII polypeptide and the activity of the wild type human Factor VIIa is at least about 2.0 when tested in the “In Vitro Proteolysis Assay”. In a further embodiment the ratio between the activity of the Factor VII polypeptide and the activity of the wild type human Factor VIIa is at least about 4.0 when tested in the “In Vitro Proteolysis Assay”. In a further embodiment the ratio between the activity of the Factor VII polypeptide and the activity of the wild type human Factor VIIa is at least about 8.0 when tested in the “In Vitro Proteolysis Assay”.

The present invention is suitable for Factor VII/VIIa variants with increased activity compared to wild type human Factor VIIa. Factor VII/VIIa variants with increased activity may be found by testing in suitable assays described in the following. These assays can be performed as a simple preliminary in vitro test. Thus, the section “assays” discloses a simple test (entitled “In Vitro Hydrolysis Assay”) for the activity of Factor VIIa variants of the invention. Based thereon, Factor VIIa variants which are of particular interest are such variants where the ratio between the activity of the variant and the activity of wild type human Factor VII is above 1.0, e.g. at least about 1.25, preferably at least about 2.0, such as at least about 3.0 or, even more preferred, at least about 4.0 when tested in the “In Vitro Hydrolysis Assay”.

The activity of the variants can also be measured using a physiological substrate such as factor X (“In Vitro Proteolysis Assay”) (see under “assays”), suitably at a concentration of 100-1000 nM, where the factor Xa generated is measured after the addition of a suitable chromogenic substrate (e.g. S-2765). In addition, the activity assay may be run at physiological temperature.

The ability of the Factor VIIa variants to generate thrombin can also be measured in an assay comprising all relevant coagulation factors and inhibitors at physiological concentrations (minus factor VIII when mimicking hemophilia A conditions) and activated platelets (as described on p. 543 in Monroe et al. (1997) Brit. J. Haematol. 99, 542-547 which is hereby incorporated as reference).

The term “identity” as known in the art, refers to a relationship between the sequences of two or more polypeptide molecules or two or more nucleic acid molecules, as determined by comparing the sequences. In the art, “identity” also means the degree of sequence relatedness between nucleic acid molecules or between polypeptides, as the case may be, as determined by the number of matches between strings of two or more nucleotide residues or two or more amino acid residues. “Identity” measures the percent of identical matches between the smaller of two or more sequences with gap alignments (if any) addressed by a particular mathematical model or computer program (i.e., “algorithms”).

The term “similarity” is a related concept, but in contrast to “identity”, refers to a sequence relationship that includes both identical matches and conservative substitution matches. If two polypeptide sequences have, for example, (fraction (10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction (15/20))). Therefore, in cases where there are conservative substitutions, the degree of similarity between two polypeptides will be higher than the percent identity between those two polypeptides.

The term “isolated polypeptide” refers to a polypeptide of the present invention that (1) has been separated from at least about 50 percent of polynucleotides, lipids, carbohydrates or other materials (i.e., contaminants) with which it is naturally associated, (2) is not covalently linked to all or a portion of a polypeptide to which the “isolated polypeptide” is linked in nature, (3) is operably linked covalently to a polypeptide to which it is not covalently linked in nature, or (4) does not occur in nature. Preferably, the isolated polypeptide is substantially free from any other contaminating polypeptides or other contaminants that are found in its natural environment which would interfere with its therapeutic, diagnostic, prophylactic or research use.

Conservative modifications to the amino acid sequence of wild type human FVII (and the corresponding modifications to the encoding nucleotides) will produce FVII polypeptides having functional and chemical characteristics similar to those of naturally occurring FVII polypeptide. In contrast, substantial modifications in the functional and/or chemical characteristics of FVII polypeptides may be accomplished by selecting substitutions in the amino acid sequence of wild type human FVII that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643:55-67; Sasaki et al., 1998, Adv. Biophys. 35:1-24, which discuss alanine scanning mutagenesis).

Identity and similarity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually (fraction ( 1/10)) times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a polypeptide sequence comparison include the following: Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following: Algorithm: Needleman et al., J. Mol. Biol., 48:443-453 (1970); Comparison matrix: matches=+10, mismatch=0, Gap Penalty: 50, Gap Length Penalty: 3.

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

Factor VII variants having a substantially modified biological activity relative to wild-type Factor VII include, without limitation, Factor VII variants that exhibit TF-independent Factor X proteolytic activity.

As described above the present invention further relates to a method for the treatment of bleeding episodes in a subject or for the enhancement of the normal haemostatic system, the method comprising administering to a subject in need thereof a therapeutically or prophylactically effective amount of a composition comprising a Factor VII polypeptide.

It is to be understood, that the subject is treated for the same bleeding or series of bleedings.

The term “enhancement of the normal haemostatic system” means an enhancement of the ability to generate thrombin.

As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation Factors XI or VII), clotting factor inhibitors, defective platelet function, thrombocytopenia or von Willebrand's disease.

The term “bleeding episodes” is meant to include uncontrolled and excessive bleeding which is a major problem both in connection with surgery and other forms of tissue damage. Uncontrolled and excessive bleeding may occur in subjects having a normal coagulation system and subjects having coagulation or bleeding disorders. Clotting factor deficiencies (haemophilia A and B, deficiency of coagulation factors XI or VII) or clotting factor inhibitors may be the cause of bleeding disorders. Excessive bleedings also occur in subjects with a normally functioning blood clotting cascade (no clotting factor deficiencies or -inhibitors against any of the coagulation factors) and may be caused by a defective platelet function, thrombocytopenia or von Willebrand's disease. In such cases, the bleedings may be likened to those bleedings caused by haemophilia because the haemostatic system, as in haemophilia, lacks or has abnormal essential clotting “compounds” (such as platelets or von Willebrand factor protein) that causes major bleedings. In subjects who experience extensive tissue damage in association with surgery or vast trauma, the normal haemostatic mechanism may be overwhelmed by the demand of immediate haemostasis and they may develop bleeding in spite of a normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

In one embodiment of the invention, the bleeding is associated with haemophilia. In another embodiment, the bleeding is associated with haemophilia with acquired inhibitors. In another embodiment, the bleeding is associated with thrombocytopenia. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with haemorrhagic gastritis. In another embodiment, the bleeding is profuse uterine bleeding. In another embodiment, the bleeding is occurring in organs with a limited possibility for mechanical haemostasis. In another embodiment, the bleeding is occurring in the brain, inner ear region or eyes. In another embodiment, the bleeding is associated with the process of taking biopsies. In another embodiment, the bleeding is associated with anticoagulant therapy.

The term “subject” as used herein is intended to mean any animal, in particular mammals, such as humans, and may, where appropriate, be used interchangeably with the term “patient”.

In the present specification and claims, the term “amino acid” denotes any molecule having the formula COOH—CR—NH₃, i.e. the term includes within its scope both naturally occurring and non-naturally occurring L- and D-amino acids. In most cases, amino acid manipulation discussed herein will involve use of naturally occurring L-amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The Factor VII polypeptides described herein may be produced by means of recombinant nucleic acid techniques. In general, a cloned wild-type Factor VII nucleic acid sequence is modified to encode the desired protein. This modified sequence is then inserted into an expression vector, which is in turn transformed or transfected into host cells. In one embodiment, higher eukaryotic cells, in particular cultured mammalian cells, are used as host cells. The complete nucleotide and amino acid sequences for human Factor VII are known (see U.S. Pat. No. 4,784,950, where the cloning and expression of recombinant human Factor VII is described). The bovine Factor VII sequence is described in Takeya et al., J. Biol. Chem. 263:14868-14872 (1988)).

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of Factor VII, one may introduce the alteration(s) of choice. Likewise, procedures for preparing a DNA construct using polymerase chain reaction using specific primers are well known to persons skilled in the art (cf. PCR Protocols, 1990, Academic Press, San Diego, Calif., USA).

The nucleic acid construct encoding the Factor VII polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Laboratory, Cold Spring Harbor, N.Y., 1989).

The nucleic acid construct encoding the Factor VII polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesiser, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct is preferably a DNA construct. DNA sequences for use in producing Factor VII polypeptides according to the present invention will typically encode a pre-pro polypeptide at the amino-terminus of Factor VII to obtain proper posttranslational processing (e.g. gamma-carboxylation of glutamic acid residues) and secretion from the host cell. The pre-pro polypeptide may be that of Factor VII or another vitamin K-dependent plasma protein, such as Factor IX, Factor X, prothrombin, protein C or protein S. As will be appreciated by those skilled in the art, additional modifications can be made in the amino acid sequence of the Factor VII polypeptides where those modifications do not significantly impair the ability of the protein to act as a coagulant. For example, the Factor VII polypeptides can also be modified in the activation cleavage site to inhibit the conversion of zymogen Factor VII into its activated two-chain form, as generally described in U.S. Pat. No. 5,288,629.

Expression vectors for use in expressing Factor VIIa variants will comprise a promoter capable of directing the transcription of a cloned gene or cDNA. Preferred promoters for use in cultured mammalian cells include viral promoters and cellular promoters. Viral promoters include the SV40 promoter (Subramani et al., Mol. Cell. Biol. 1:854-864, 1981) and the CMV promoter (Boshart et al., Cell 41:521-530, 1985). A particularly preferred viral promoter is the major late promoter from adenovirus 2 (Kaufman and Sharp, Mol. Cell. Biol. 2:1304-1319, 1982). Cellular promoters include the mouse kappa gene promoter (Bergman et al., Proc. Natl. Acad. Sci. USA 81:7041-7045, 1983) and the mouse VH promoter (Loh et al., Cell 33:85-93, 1983). A particularly preferred cellular promoter is the mouse metallothionein-1 promoter (Palmiter et al., Science 222:809-814, 1983). Expression vectors may also contain a set of RNA splice sites located downstream from the promoter and upstream from the insertion site for the Factor VII sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 E1b region, the human growth hormone gene terminator (DeNoto et al. Nucl. Acids Res. 9:3719-3730, 1981) or the polyadenylation signal from the human Factor VII gene or the bovine Factor VII gene. The expression vectors may also include a noncoding viral leader sequence, such as the adenovirus 2 tripartite leader, located between the promoter and the RNA splice sites; and enhancer sequences, such as the SV40 enhancer.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. If, on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells. After the cells have taken up the DNA, they are grown in an appropriate growth medium, typically for 1-2 days, to begin expressing the gene of interest. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991). Growth media generally include a carbon source, a nitrogen source, essential amino acids, essential sugars, vitamins, salts, phospholipids, proteins and growth factors. For production of gamma-carboxylated Factor VII polypeptides, the medium will contain vitamin K, preferably at a concentration of about 0.1 mg/ml to about 5 mg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the desired Factor VII polypeptide.

Preferred mammalian cell lines include the CHO (ATCC CCL 61), COS-1 (ATCC CRL 1650), baby hamster kidney (BHK) and HEK293 (e.g., ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982), hereinafter referred to as BHK 570 cells. The BHK 570 cell line is available from the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1) and DUKX cells (Urlaub and Chasin, Proc. Natl. Acad. Sci. USA 77:4216-4220, 1980).

Transgenic animal technology may be employed to produce the Factor VII polypeptides of the invention. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and biochemically well characterized. Furthermore, the major milk proteins are present in milk at high concentrations (typically from about 1 to 15 g/l).

From a commercial point of view, it is clearly preferable to use as the host a species that has a large milk yield. While smaller animals such as mice and rats can be used (and are preferred at the proof of principle stage), it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk (see, for example, WO 88/00239 for a comparison of factors influencing the choice of host species). It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489), beta lactoglobulin, a lactalbumin, and whey acidic protein. The beta lactoglobulin (BLG) promoter is preferred. In the case of the ovine beta lactoglobulin gene, a region of at least the proximal 406 bp of 5′ flanking sequence of the gene will generally be used, although larger portions of the 5′ flanking sequence, up to about 5 kbp, are preferred, such as a ˜4.25 kbp DNA segment encompassing the 5′ flanking promoter and non coding portion of the beta lactoglobulin gene (see Whitelaw et al., Biochem. J. 286: 31 39 (1992)). Similar fragments of promoter DNA from other species are also suitable.

Other regions of the beta lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836 840 (1988); Palmiter et al., Proc. Natl. Acad. Sci. USA 88: 478 482 (1991); Whitelaw et al., Transgenic Res. 1: 3 13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta lactoglobulin gene, is preferred. One such region is a DNA segment that provides for intron splicing and RNA polyadenylation from the 3′ non coding region of the ovine beta lactoglobulin gene. When substituted for the natural 3′ non coding sequences of a gene, this ovine beta lactoglobulin segment can both enhance and stabilize expression levels of the protein or polypeptide of interest. Within other embodiments, the region surrounding the initiation ATG of the variant Factor VII sequence is replaced with corresponding sequences from a milk specific protein gene. Such replacement provides a putative tissue specific initiation environment to enhance expression. It is convenient to replace the entire variant Factor VII pre pro and 5′ non coding sequences with those of, for example, the BLG gene, although smaller regions may be replaced.

For expression of Factor VII polypeptides in transgenic animals, a DNA segment encoding variant Factor VII is operably linked to additional DNA segments required for its expression to produce expression units. Such additional segments include the above mentioned promoter, as well as sequences that provide for termination of transcription and polyadenylation of mRNA. The expression units will further include a DNA segment encoding a secretory signal sequence operably linked to the segment encoding modified Factor VII. The secretory signal sequence may be a native Factor VII secretory signal sequence or may be that of another protein, such as a milk protein (see, for example, von Heijne, Nucl. Acids Res. 14: 4683 4690 (1986); and Meade et al., U.S. Pat. No. 4,873,316, which are incorporated herein by reference).

Construction of expression units for use in transgenic animals is conveniently carried out by inserting a variant Factor VII sequence into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of a variant Factor VII polypeptide; thereby creating a gene fusion that includes the expression control sequences of the milk protein gene. In any event, cloning of the expression units in plasmids or other vectors facilitates the amplification of the variant Factor VII sequence. Amplification is conveniently carried out in bacterial (e.g. E. coli) host cells, thus the vectors will typically include an origin of replication and a selectable marker functional in bacterial host cells. The expression unit is then introduced into fertilized eggs (including early stage embryos) of the chosen host species. Introduction of heterologous DNA can be accomplished by one of several routes, including microinjection (e.g. U.S. Pat. No. 4,873,191), retroviral infection (Jaenisch, Science 240: 1468 1474 (1988)) or site directed integration using embryonic stem (ES) cells (reviewed by Bradley et al., Bio/Technology 10: 534 539 (1992)). The eggs are then implanted into the oviducts or uteri of pseudopregnant females and allowed to develop to term. Offspring carrying the introduced DNA in their germ line can pass the DNA on to their progeny in the normal, Mendelian fashion, allowing the development of transgenic herds. General procedures for producing transgenic animals are known in the art (see, for example, Hogan et al., Manipulating the Mouse Embryo: A Laboratory Manual, Cold Spring Harbor Laboratory, 1986; Simons et al., Bio/Technology 6: 179 183 (1988); Wall et al., Biol. Reprod. 32: 645 651 (1985); Buhler et al., Bio/Technology 8: 140 143 (1990); Ebert et al., Bio/Technology 9: 835 838 (1991); Krimpenfort et al., Bio/Technology 9: 844 847 (1991); Wall et al., J. Cell. Biochem. 49: 113 120 (1992); U.S. Pat. No. 4,873,191; U.S. Pat. No. 4,873,316; WO 88/00239, WO 90/05188, WO 92/11757; and GB 87/00458). Techniques for introducing foreign DNA sequences into mammals and their germ cells were originally developed in the mouse (see, e.g., Gordon et al., Proc. Natl. Acad. Sci. USA 77: 7380 7384 (1980); Gordon and Ruddle, Science 214: 1244 1246 (1981); Palmiter and Brinster, Cell 41: 343 345 (1985); Brinster et al., Proc. Natl. Acad. Sci. USA 82: 4438 4442 (1985); and Hogan et al. (ibid.)). These techniques were subsequently adapted for use with larger animals, including livestock species (see, e.g., WO 88/00239, WO 90/05188, and WO 92/11757; and Simons et al., Bio/Technology 6: 179 183 (1988)). To summarize, in the most efficient route used to date in the generation of transgenic mice or livestock, several hundred linear molecules of the DNA of interest are injected into one of the pro nuclei of a fertilized egg according to established techniques. Injection of DNA into the cytoplasm of a zygote can also be employed.

Production in transgenic plants may also be employed. Expression may be generalized or directed to a particular organ, such as a tuber (see, Hiatt, Nature 344:469 479 (1990); Edelbaum et al., J. Interferon Res. 12:449 453 (1992); Sijmons. et al., Bio/Technology 8:217 221 (1990); and EP 0 255 378).

The Factor VII polypeptides of the invention are recovered from cell culture medium or milk. The Factor VII polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). Preferably, they may be purified by affinity chromatography on an anti-Factor VII antibody column. The use of calcium-dependent monoclonal antibodies is described by Wakabayashi et al., J. Biol. Chem. 261:11097-11108, (1986) and Thim et al., Biochemistry 27: 7785-7793, (1988). Additional purification may be achieved by conventional chemical purification means, such as high performance liquid chromatography. Other methods of purification, including barium citrate precipitation, are known in the art, and may be applied to the purification of the novel Factor VII polypeptides described herein (see, for example, Scopes, R., Protein Purification, Springer-Verlag, N.Y., 1982).

For the purpose of recombinant expression of Factor VII polypeptides in prokaryotic cells, the nucleic acid fragments encoding the Factor VII polypeptides will normally be inserted in suitable vectors to form cloning or expression vectors carrying the nucleic acid fragments of the invention. Details concerning the construction of these vectors of the invention will be discussed in context of transformed cells and microorganisms below. The vectors can be in the form of plasmids, phages, cosmids, or mini-chromosomes. Preferred cloning and expression vectors used in the invention are capable of autonomous replication, thereby enabling high copy-numbers for the purposes of high-level expression or high-level replication for subsequent cloning.

The general outline of a vector for use in the of the invention comprises the following features in the 5′-3′ direction and in operable linkage: a promoter for driving expression of the nucleic acid fragment encoding the Factor VII polypeptides, optionally a nucleic acid sequence encoding a leader peptide enabling secretion (to the extracellular phase or, where applicable, into the periplasm) of or integration into the membrane of the Factor VII polypeptides, the nucleic acid fragment of the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome.

The vectors of the invention are used to transform host cells to produce the Factor VII polypeptides. Such transformed cells can be cultured cells or cell lines used for propagation of the nucleic acid fragments and vectors encoding the second polyamino acid or used for recombinant production of Factor VII polypeptides.

In one embodiment, the transformed cells of the invention are microorganisms such as bacteria (such as the species Escherichia [e.g. E. coli], Bacillus [e.g. Bacillus subtilis], Rhodococcus, Streptomycetes, Actinomycetes, Corynebacteria, Pseudomonas, Erwinia or Salmonella; yeasts (such as Saccharomyces cerevisiae and Picchia); insect cells; and protozoans. Alternatively, the transformed cells are derived from a multicellular organism such as a fungi (Penicillium, Fusarium, Aspergillus, Podospora, Neurospora) or a plant cell.

In one embodiment of the invention, the Factor VII polypeptide is non-glycosylated.

Prokaryotic host cells, including strains of the bacteria Escherichia coli, Bacillus and other genera are the most useful host cells within the present invention. Techniques for transforming these hosts and expressing foreign DNA sequences cloned therein are well known in the art (see, e.g., Sambrook et al., ibid.). When expressing DNA encoding a Factor VII polypeptides in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the polypeptide, thereby obviating the need for denaturation and refolding. Methods for producing heterologous disulfide bond-containing polypeptides in bacterial cells are disclosed by Georgiou et al., U.S. Pat. No. 6,083,715.

For the purposes of cloning and/or optimized expression it is preferred that the transformed cell is capable of replicating the nucleic acid fragment encoding the Factor VII polypeptides. To ultimately produce the Factor VII polypeptides, transformed cells must express the nucleic acid fragment encoding the first polyamino acid. It is convenient, although far from essential, that the expression product is either exported out into the culture medium or carried on the surface of the transformed cell.

When an effective producer cell has been identified it is preferred, on the basis thereof, to establish a stable cell line which carries the which expresses the nucleic acid fragment encoding the Factor VII polypeptides. Preferably, this stable cell line secretes or carries on its surfaces the Factor VII polypeptides, thereby facilitating purification thereof. In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species. The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available. For expression in Saccharomyces, the plasmid YRp7, for example, is commonly used. This plasmid already contains the trpI gene which provides a selection marker for a mutant strain of yeast lacking the ability to grow in tryptophan for example ATCC No. 44076 or PEP4-1. The presence of the trpI lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

For therapeutic purposes it is preferred that the Factor VII polypeptides of the invention are substantially pure. Thus, in a preferred embodiment of the invention the Factor VII polypeptides of the invention is purified to at least about 90 to 95% homogeneity, preferably to at least about 98% homogeneity. Purity may be assessed by e.g. gel electrophoresis and amino-terminal amino acid sequencing.

The Factor VII polypeptide is cleaved at its activation site in order to convert it to its two-chain form. Activation may be carried out according to procedures known in the art, such as those disclosed by Osterud, et al., Biochemistry 11:2853-2857 (1972); Thomas, U.S. Pat. No. 4,456,591; Hedner and Kisiel, J. Clin. Invest. 71:1836-1841 (1983); or Kisiel and Fujikawa, Behring Inst. Mitt. 73:29-42 (1983). Alternatively, as described by Bjoern et al. (Research Disclosure, 269 September 1986, pp. 564-565), Factor VII may be activated by passing it through an ion-exchange chromatography column, such as Mono Q (Pharmacia fine Chemicals) or the like. The resulting activated Factor VII polypeptide may then be formulated and administered as described below.

Assays

Assay 1:

In Vitro Hydrolysis Assay

Wild type (native) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMax® 340 plate reader (Molecular Devices, USA). The absorbance developed during a 20-minute incubation, after subtraction of the absorbance in a blank well containing no enzyme, is used to calculate the ratio between the activities of variant and wild-type Factor VIIa:
Ratio=(A405 nm Factor VIIa variant)/(A405 nm Factor VIIa wild-type).
Assay 2:
In Vitro Proteolysis Assay
Wild type (native) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl₂ and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax® 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIa:
Ratio=(A405 nm Factor VIIa variant)/(A405 nm Factor VIIa wild-type).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### BRIEF DESCRIPTION OF THE DRAWINGS

FIG. 1. The amino acid sequence of wild type human coagulation Factor VII.

### EXAMPLES

### Example 1

Production of FVII polypeptides according to the invention by Cathepsin G cleavage for removal of amino acid residues 1-44 relative to the amino acid sequence of SEQ ID NO:1.

DNA constructs encoding FVII polypeptides comprising one or more amino acid substitutions relative to the amino acid sequence of SEQ ID NO:1 may be prepared by site-directed mutagenesis using a supercoiled, double stranded DNA vector with an insert of interest and two synthetic primers containing the desired mutation as described in Published international patent applications WO 01/83725, WO 02/22776, WO 03/027147, WO 02/077218, and WO 03/037932 and Danish patent application PA 2002 01423. Briefly oligonucleotide primers, each complementary to opposite strands of the vector, are extended during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA.

Procedures for preparing a DNA construct using polymerase chain reaction using specific primers are well known to persons skilled in the art (cf. PCR Protocols, 1990, Academic Press, San Diego, Calif., USA).

FVII polypeptides comprising one or more amino acid substitutions relative to the amino acid sequence of SEQ ID NO:1 may be prepared by transfection in BHK cells essentially as previously described (Thim et al. (1988) Biochemistry 27, 7785-7793; Persson and Nielsen (1996) FEBS Lett. 385, 241-243) to obtain expression of the FVII polypeptide variant.

The FVII polypeptides comprising one or more amino acid substitutions relative to the amino acid sequence of SEQ ID NO:1 may be purified as follows:

Conditioned medium is loaded onto a 25-ml column of Q Sepharose Fast Flow (Pharmacia Biotech) after addition of 5 mM EDTA, 0.1% Triton X-100 and 10 mM Tris, adjustment of pH to 8.0 and adjustment of the conductivity to 10-11 mS/cm by adding water.

Elution of the protein is accomplished by stepping from 10 mM Tris, 50 mM NaCl, 0.1% Triton X-100, pH 8.0 to 10 mM Tris, 50 mM NaCl, 25 mM CaCl2, 0.1% Triton X-100, pH 8.0. The fractions containing the FVII polypeptide variant are pooled and applied to a 25-ml column containing monoclonal antibody F1A2 (Novo Nordisk, Bagsvaerd, Denmark) coupled to CNBr-activated Sepharose 4B (Pharmacia Biotech).

The column is equilibrated with 50 mM Hepes, pH 7.5, containing 10 mM CaCl2,100 mM NaCl and 0.02% Triton X-100. After washing with equilibration buffer and equilibration buffer containing 2 M NaCl, bound material is eluted with equilibration buffer containing 10 mM EDTA instead of CaCl2. Before use or storage, excess CaCl2 over EDTA is added or the FVII polypeptide variant is transferred to a Ca2+-containing buffer. The yield of each step is followed by factor VII ELISA measurements and the purified protein is analyzed by SDS-PAGE.

Following purification of the FVII polypeptides comprising one or more amino acid substitutions relative to the amino acid sequence of SEQ ID NO:1 the polypeptide is treated with Cathepsin G cleavage for removal of amino acid residues 1-44 relative to the amino acid sequence of SEQ ID NO:1 as described in Nicolaisen et al. (1992) FEBS Lett. 306, 157-160.

### Example 2

Production of FVII polypeptides according to the invention by removal of DNA sequence encoding functional lipid membrane binding domain amino acid residues from DNA constructs encoding FVII polypeptides.

Construction of DNA encoding truncated versions of FVII polypeptides as exemplified by FVII polypeptides encompassing residues 42-406 (FVII-(42-406)) and 83-406 (FVII-(83-406)): DNA constructs encoding N-terminally truncated versions of FVII polypeptides were prepared by site-directed mutagenesis using two synthetic primers bridging the desired deletion and a supercoiled, double stranded DNA vector with insert of unprocessed human FVII, i.e. comprising the FVII leader peptide (as described in U.S. Pat. No. 4,784,950) followed by the mature FVII sequence (FIG. 1, SEQ ID NO:1). Each primer was designed to simultaneously anneal to the two regions of the DNA vector flanking the desired deletion. Since deletions encompassed the N-terminal part of mature FVII, one flanking region (underlined) was located in the leader-peptide sequence.

| | |
|---|---|
| For FVII-(42-406): | |
| 5′-GGCGTCCTGCACCGGCGCCGGCGCATTTCTTACA | (SEQ ID NO:2) |
| GTGATGGGGACCAGTGTGCCTC-3′ | |
| 5′-GAGGCACACTGGTCCCCATCACTGTAAGAAATGC | (SEQ ID NO:3) |
| GCCGGCGCCGGTGCAGGACGCC-3′ | |
| For FVII-(83-406): | |
| 5′-GGCGTCCTGCACCGGCGCCGGCGCACGCACAAGG | (SEQ ID NO:4) |
| ATGACCAGCTGATCTGTGTG-3′ | |
| 5′-CACACAGATCAGCTGGTCATCCTTGTGCGTGCGC | (SEQ ID NO:5) |
| CGGCGCCGGTGCAGGACGCC-3′ | |

The oligonucleotide primers, each complementary to opposite strands of the vector insert, were extended during temperature cycling by means of Pfu DNA polymerase. On incorporation of the primers, a mutated plasmid containing staggered nicks was generated. Following temperature cycling, the product was treated with Dpnl which is specific for methylated and hemimethylated DNA to digest the parental DNA template and to select for mutation-containing synthesized DNA. Procedures for preparing a DNA construct using polymerase chain reaction using specific primers are well known to persons skilled in the art (cf. PCR Protocols, 1990, Academic Press, San Diego, Calif., USA).

### Example 3

Demonstration of the procoagulant activity of a FVII polypeptide lacking residues 1-44.

Anti-FVIII antibody (final concentration 10 human Bethesda units/ml) was added to citrated human blood from a normal donor to induce a haemophilia A-like condition. Full-length or des(1-44)-FVIIa was added to this blood to a final concentration of 25 or 100 nM. Innovin (relipidated recombinant human tissue factor, final dilution 1:50000) and t-PA (final concentration 1.8 nM) was also added. Blood clotting was initiated by the addition of buffer containing CaCl₂, and the development of a fibrin clot was monitored using thrombelastography. The procoagulant activity of des(1-44)-FVIIa was similar to that of FVIIa. For instance, the presence of 100 nM des(1-44)-FVIIa gave clotting times of 490 and 455 seconds in two samples, whereas 100 nM FVIIa gave clotting times of 390 and 410 seconds. All four samples were based on the same donor blood. The rate by which the clot grew, represented by the so-called angle value, was also similar in the two des(1-44)-FVIIa samples (59 and 620) and in the two FVIIa samples (57 and 61°). The maximal clot strength, represented by the maximal amplitude value, was between 51 and 54 mm in the four samples, i.e. similar for FVIIa and des(1-44)-FVIIa. The FVIIa variant V158D/E296V/M298Q-FVIIa also displayed similar results in its full-length and des(1-44) forms when tested at 1 nM.

1. A Factor VII (“FVII”) variant that is (a) free of GLA residues and (b) comprises an amino acid sequence that differs from human Factor VII (SEQ ID NO:1) by one or more substitutions of amino acid residues relative to SEQ ID NO:1 in one or more positions selected from K157, V158, E296, M298, L305, M306, D309, S314, D334, S336, K337, and F374 of SEQ ID NO:1.

2. The Factor VII variant according to claim 1, wherein K157 is substituted.

3. The Factor VII variant according to claim 1, wherein V158 is substituted.

4. The Factor VII variant according to claim 1, wherein E296 is substituted.

5. The Factor VII variant according to claim 1, wherein M298 is substituted.

6. The Factor VII variant according to claim 1, wherein L305 is substituted.

7. The Factor VII variant according to claim 1, wherein M306 is substituted.

8. The Factor VII variant according to claim 1, wherein D309 is substituted.

9. The Factor VII variant according to claim 1, wherein S314 is substituted.

10. The Factor VII variant according to claim 1 wherein D334 is substituted.

11. The Factor VII variant according to claim 1, wherein S336 is substituted.

12. The Factor VII variant according to claim 1, wherein K337 is substituted.

13. The Factor VII variant according to claim 1 wherein F374 is substituted.

14. The Factor VII variant according to claim 1, wherein at least one additional amino acid residue in the protease domain is substituted relative to human Factor VII.

15. The Factor VII variant according to claim 14, wherein at least one amino acid corresponding to an amino acid at a position selected from 159-170 of SEQ ID NO:1 is substituted.

16. The Factor VII variant according to claim 14, wherein at least one amino acid corresponding to an amino acid at a position selected from 290-304 of SEQ ID NO:1 has been replaced with any other amino acid.

17. The Factor VII variant according to claim 16, wherein R304 is replaced by an amino acid selected from the group consisting of Tyr, Phe, Leu, and Met.

18. The Factor VII variant according to claim 14, wherein at least one amino acid corresponding to an amino acid at a position selected from 307-312 of SEQ ID NO:1 is substituted.

19. The Factor VII variant according to claim 14, wherein at least one amino acid corresponding to an amino acid at a position selected from 330-339 of SEQ ID NO:1 is substituted.

20. The Factor VII variant according to claim 14, wherein A274 is substituted.

21. The Factor VII variant according to claim 20, wherein A274 has been replaced by an amino acid selected from the group consisting of Met, Leu, Lys, and Arg.

22. The Factor VII variant according to claim 2, wherein K1157 is replaced by an amino acid selected from the group consisting of Gly, Val, Ser, Thr, Asn, Gin, Asp, and Glu.

23. The Factor VII variant according to claim 3, wherein V1158 is replaced by an amino acid selected from the group consisting of Ser, Thr, Asn, Gln, Asp, and Glu.

24. The Factor VII variant according to claim 4, wherein E296 is replaced by an amino acid selected from the group consisting of Arg, Lys, Ile, Leu and Val.

25. The Factor VII variant according to claim 5, wherein M298 is replaced by an amino acid selected from the group consisting of Lys, Arg, Gln, and Asn.

26. The Factor VII variant according to claim 6, wherein L305 is replaced by an amino acid selected from the group consisting of Val, Tyr and Ile.

27. The Factor VII variant according to claim 7, wherein M306 is replaced by an amino acid selected from the group consisting of Asp, and Asn.

28. The Factor VII variant according to claim 8, wherein D309 is replaced by an amino acid selected from the group consisting of Ser, and Thr.

29. The Factor VII variant according to claim 9, wherein S314 is replaced by an amino acid selected from the group consisting of Gly, Lys, Gln and Glu.

30. The Factor VII variant according to claim 10, wherein D334 is replaced by an amino acid selected from the group consisting of Gly, and Glu.

31. The Factor VII variant according to claim 11, wherein S336 is replaced by an amino acid selected from the group consisting of Gly, and Glu.

32. The Factor VII variant according to claim 12, wherein K337 is replaced by an amino acid selected from the group consisting of Ala, Gly, Val, Ser, Thr, Asn, Gln, Asp, and Glu.

33. The Factor VII variant according to claim 13, wherein said F374 has been replaced by an amino acid selected from the group consisting of Pro, and Tyr.

34. The Factor VII variant according to claim 33, wherein said F374 has been replaced by Tyr.

35. The Factor VII variant according to claim 1, wherein the Factor VII variant lacks residues 1-38 of SEQ ID NO:1.

36. The Factor VII variant according to claim 35, wherein the Factor VII variant lacks residues 1-44 of SEQ ID NO:1.

37. The Factor VII variant according to claim 36, wherein the Factor VII variant lacks residues 1-152 of SEQ ID NO:1.

39. The Factor VII variant according to claim 1, wherein the ratio between the proteolytic activity of the Factor VII variant and the proteolytic activity of human Factor VIIa is at least about 1.25.

40. A polynucleotide comprising a sequence that encodes a Factor VII variant according to claim 1.

41. A pharmaceutically acceptable composition comprising a FVII polypeptide that is essentially free of GLA residues and and, optionally, a pharmaceutically acceptable carrier.

42. The composition of claim 41, wherien the FVII polypeptide is a FVII variant according to claim 1.

43. The composition of claim 41, wherein the FVII polypeptide is a wild-type human FVII that is essentially free of GLA residues.

44. A method for treating a bleeding disorder, treating a bleeding episode, or enhancing hemostasis in a subject comprising administering a therapeutically or prophylactically effective amount of a FVII polypeptide that is essentially free of GLA residues to a subject in need thereof.

45. The method of claim 44, wherein the method comprises administering a therapeutically or prophylactically effective amount of a FVII variant according to claim 1 to the subject.
